(19) United States
(12) Patent Application Publication
(10) Pub. No.: US 2011/0040389 A1
(43) Pub. Date: Feb. 17, 2011

(54) HYDROXYAPATITE, BIOCOMPATIBLE GLASS AND SILICON-BASED BONE SUBSTITUTE, PRODUCTION PROCESS AND APPLICATIONS THEREOF

(76) Inventors: José Domingos Da Silva Santos, Porto (PT); Maria Ascensão Ferreira Da Silva Lopes, Porto (PT); Cláudia Manuela Da Cunha Ferreira Botelho, Porto (PT)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(21) Appl. No.: 12/936,670

(22) PCT Filed: Apr. 7, 2008
(86) PCT No.: PCT/PT08/00014

§ 371 Date: Oct. 6, 2010

(51) Int. Cl. A61F 2/28

(52) U.S. Cl. 623/23.61

(57) ABSTRACT

The disclosed invention is based on hydroxyapatite, biocompatible glass and silicon, aiming to develop an improved bone substitute, which presents higher mechanical resistance, bioactivity and osteoregeneration, and susceptible of being used in several medical and surgical fields, with application in the treatment of bone disease caused by trauma or genetic factors, as osteoconductive support for cellular growth. The abovementioned bone substitute comprises hydroxyapatite, a biocompatible glass of the P₂O₅—CaO system in a percentage up to 10 wt % relatively to the hydroxyapatite weight, and a silicon source in a concentration up to 10 wt % relatively to hydroxyapatite and biocompatible glass weight. The preparation process of the disclosed bone substitute consists of liquid phase sintering of a homogeneous mixture of hydroxyapatite, biocompatible glass and silicon source preferentially within a temperature range of 1100-13500 C, which allows glass melting and fusion throughout hydroxyapatite structure leading to the occurrence of several ionic substitutions.

### FIELD OF THE INVENTION

The present invention refers to the development of a medical device, namely, a hydroxyapatite, biocompatible glass and silicon-based synthetic bone substitute, with several applications in the medical field.

### BACKGROUND OF INVENTION

Bone defects resulting from trauma, tumour resection, non-union of fractures and congenital malformations are common clinical problems. Nowadays, several bone grafts which are currently being used include autograft (tissue from another location of the body of the same individual), allograft (tissue from different individuals of the same species), xenograft (tissue implanted from a different species), and synthetic bone graft (biomaterials). Though autograft is the considered most suitable for the majority of medical applications, it requires at least a second surgery for graft harvesting, usually from fibula, iliac crest or radius from the patient. This second surgical procedure causes donor site morbidity associated with haemorrhage, infection and pain. Concerning allografts and xenografts, both present high immunological risk with potential infectious disease transmission.

Due to the abovementioned motives, the synthetic bone graft development and applications have recently expanded. There have been several attempts to develop a synthetic bone graft that substitutes human bone tissue, namely, hydroxyapatite and tricalcium phosphate. These biomaterials comprise the most employed group of bone substitutes in osteoregenerative medicine, since their structure is similar to the bone mineral phase, and are characterized as biocompatible, bioactive and osteoconductive.

Silicon is the third most abundant trace element in the human body, with the highest levels found in connective tissue, namely in bone. The bioavailability of silicon is critical for the development and structural integrity of connective tissue in mammalian systems¹. There are evidences concerning silicon's role during the deposition of extracellular matrix, particularly, in the hydroxylation of the intracellular proline during procollagen synthesis, in forming crosslinks at hydroxylysine/lysine sites and in stabilising the glycosaminoglycan network. The high silicon concentration observed in numerous extracellular matrixes implies that this element plays an important role as a biological cross-linking agent, which contributes to the arquitecture and resilience of connective tissue.

Studies carried out by Carlisle^{1,2} have shown the importance of silicon in bone formation and mineralization. Silicon levels up to 0.5 wt % were observed in these areas, suggesting that this element has an important role in the calcification process. Similar studies by Schwarz and Milne³, have shown that silicon deficiency in rats resulted in skull deformations. Additional studies demonstrated benefits in delivering controlled levels of silicon to a bone defect site, resulting in enhanced bone repair through significant up-regulation of osteoblast proliferation and gene expression (including BMP-2)³.

Currently, several silicon rich materials, such as glass and glass-ceramics, have been proposed as bone graft materials due to their high bioactivity⁴. However, these materials despite containing high levels of SiO₂ (30-60 wt %), present low degradation and therefore the released silicon does not reach normal physiological levels. Nevertheless, it is proposed that the SiO₂-rich glass bioactivity is related with the role of SiO₂ or elemental silicon present in their surfaces. Silicon-substituted apatites with levels of silicon up to 10 wt % have also been proposed, and their improved bioactivity with respect to non-substituted apatites has been evidenced by in vitro and in vivo conditions⁴. However, when compared to pure phase hydroxyapatite (HA), these silicon-substituted apatites do not show any improvement in terms of mechanical properties. Furthermore, Si-HA apatites do not mimic the composition of human bone tissue, which is a composite material containing several ionic substitutions such as sodium, fluorine, magnesium and potassium.

While WO0068164⁵ discloses a sintered hydroxyapatite composite with a phosphate-based glass containing fluorine, which is the ionic species responsible for its bioactivity, the material of the present invention comprises a triphasic mixture (hydroxyapatite, alpha and beta tricalcium phosphate (TCP)) with higher bioactivity due to the addition of silicon. Moreover, relatively to the abovementioned document, the bone substitute of the present invention has higher proportions of alpha and beta-TCP secondary phases in its structure whose formation is induced by the addition of silicon. The amount of secondary phases, alpha and beta-TCP, present in the bone substitute is nevertheless highly controllable and varies according to the quantity of silicon added.

While WO9808773⁶ discloses a synthetic apatite and hydroxyapatite supplemented with silicon during chemical synthesis, resulting in a bone substitute of pure phase, the bone substitute of the present invention is obtained by means of liquid phase sintering between hydroxyapatite, biocompatible glass and silicon, and concomitant formation of the secondary phases alpha and beta-TCP, arranged in a unique microstructure which enhances its mechanical properties.

While U.S. Pat. No. 6,846,493⁷ discloses a production method of a calcium phosphate material by chemical synthesis in which the supplementation with silicon is done during the precipitation step and subsequent sinterization process is performed up to 1000° C., resulting in a bone substitute comprising hydroxyapatite, Si-TCP and beta-TCP, the bone substitute of the present invention comprises hydroxyapatite, a biocompatible glass and silicon, obtained by means of liquid phase sintering above 1100° C., resulting in the formation of the secondary phases alpha and beta-TCP, in different proportions according to the added amount of silicon, arranged in a unique microstructure, which enhances its mechanical properties.

### General Description of the Invention

The present invention refers to a synthetic bone substitute, comprehended by hydroxyapatite, biocompatible glass and silicon up to about 10 wt %, preferably up to 3 wt %, having a distinguishable microstructure of three crystallographic phases: hydroxyapatite, alpha and beta-TCP.

### 1. Bone Substitute Features

The present invention refers to a synthetic bone substitute comprising a mixture of hydroxyapatite, alpha-TCP, beta-TCP and silicon, obtained from the reaction between a biocompatible glass, silicon and hydroxyapatite, which presents an excellent osteoconductivity.

During the preparation of the bone substitute and depending on the amount and composition of the biocompatible glass, as well as on the silicon content added and on the sintering temperature, a material with a distinguishable microstructure composed by controllable percentages of HA and secondary phases alpha and beta-TCP is obtained.

The presence of alpha and beta-TCP phases, which show a higher degradation rate comparatively to hydroxyapatite, promotes the controlled release of ions, such as, silicon, fluoride, magnesium, sodium, among others, from the surface of the bone substitute to the surrounding medium, promoting the deposition of extracellular matrix of osseous connective tissue and specific activation of osteoprecursor cells thus inducing bone formation.

During sinterization of hydroxyapatite, silicon and biocompatible glass, which is performed within the temperature range of 1200-1350° C., the latter melts and diffuses within the hydroxyapatite structure leading to the occurrence of several network ionic substitutions, including silicon incorporation.

Silicon incorporation is characterized by numerous partial ionic substitutions of phosphate groups by silicate groups, or its incorporation in the hydroxyapatite structural interstices and concomitant phase composition alteration of the bone substitute. The latter phenomena depends on the silicon content added and is characterized by a diminution of hydroxyapatite percentage and consequent increase of secondary phases alpha and beta-TCP percentage (FIG. 1).

The X-ray diffraction spectra depicted on FIG. 1, describes the silicon addition effect on the phase composition of the bone substitute comprehended by HA and 2.5 wt % of a biocompatible glass, sintered at a temperature of 1300° C., demonstrating the presence of hydroxyapatite phase (Database JCPDS-ICDD File #72-1243), alpha-TCP phase (Database JCPDS-ICDD File #9-348) and beta-TCP phase (Database JCPDS-ICDD File #09-0169) on the bone substitute. Moreover, silicon addition, using a colloidal silicon source up to 3 wt %, demonstrates the coexistence of the same abovementioned phases. However, as depicted by the phase quantification (FIG. 2), silicon addition leads to a substantial increase of the alpha and beta-TCP secondary phases. Adding silicon in equal or superior amounts to 3 wt % results in other silicon-containing secondary phases appearance, such as, silica (SiO₂) and/or calcium silicates (Ca₂SiO₄ e CaSiO₃), besides alpha and beta-TCP.

The preparation of the bone substitute disclosed in the present invention allows for phase composition control and consequent biodegradability rate control resulting in a greater versatility regarding the final clinical application.

Controlled biodegradability rates results in controlled bioactivity mediated by the controlled ionic species release fundamental to osteoregeneration.

Additionally, the higher percentage of alpha and beta-TCP of the disclosed bone substitute, results in an enhanced mechanical resistance characterized by superior flexural bending strength comparatively to other hydroxyapatites.

Therefore, silicon addition up to about 10 wt %, preferably up to 3 wt %, to a mixture of hydroxyapatite and biocompatible glass, accomplishes a new bone substitute presenting physiological levels of silicon mediated-bioactivity, an improved osteointegration, a controlled biodegradability rate and enhanced mechanical properties, assuring a greater clinical outcome.

The synthetic bone substitute disclosed in the present invention aspires to clinical application in the treatment of bone diseases, due to trauma or genetic factors, as osteoconductive support (intra or extracorporeal) for cellular growth, in the form of granules, tridimensional (3D) pieces, custom-made implants and as prostheses and implant coatings or as bone cements.

Additionally, the disclosed bone substitute might be used as a composite material, comprehended by the base material associated to a biocompatible polymeric vehicle for minimal invasive surgery.

Another possible application of the disclosed bone substitute consists on a device for drug controlled release employing growth factors, as well as other drugs that influence bone growth and remodelling.

Finally, the disclosed bone substitute might be used in association with stem cells as a novel therapeutical approach in the osteoregenerative medical field.

### 2. Bone Substitute Preparation

The preparation of the disclosed bone substitute, comprehended by hydroxyapatite, biocompatible glass and silicon up to about 10 wt %, preferably up to 3 wt %, requires the mixture of a silicon source with the hydroxyapatite and the biocompatible glass.

The preparation of this bone substitute only becomes effective upon sinterization thermal treatment above 1100° C., in order to guaranty low viscosity of the added glass, thus allowing melting and distribution throughout hydroxyapatite network. After the mixture preparation of the abovementioned components, heating above 1100° C. is performed, more preferably in a temperature range within 1200° and 1350° C., with the purpose of phase composition control and bone substitute densification.

The addition of silicon to hydroxyapatite and glass might be performed using conventional silicon sources, such as colloidal silica (silica nanoparticles), tetraethylorthosilicate (TEOS, Si(OC₂H₅)₄) tetrapropylorthosilicate (TPOS, Si(OC₃H₇)₄), silicon acetate (SiC₂H₃O₂) sodium silicate (Na₂SiO₃), calcium silicate (Ca₂SiO₄) or magnesium silicate (Mg₂SiO₄), among others.

The mixture might be done, before sintering, during any step of the preparation process, through dry or wet route, for instance in a double cone mixer, in a planetary mixer or in a turbula, thus guaranteeing homogeneous mixture of the three components.

The dry mixture process requires direct addition of the solid silicon source with the hydroxyapatite and biocompatible glass powders.

The wet mixture process requires the use of aqueous or non-aqueous solvents and, depending on the employed silicon source, it might require silicon solution or suspension preparation, with the appropriate concentration, and posterior addition to the hydroxyapatite and biocompatible glass powders.

Silicon solutions or suspensions preparation might require the use of a surfactant in order to guarantee homogeneous silicon distribution on the bone substitute. In this sense, it is required the use of conventional surfactants, such as methylcellulose, saponin, polyvinyl alcohol (PVA, [CH₂CHOH]ₙ), among others.

### DESCRIPTION OF THE DRAWINGS

FIG. 1—X-ray diffraction spectra referring to the effect of silicon addition, using colloidal silica as source, on the phase composition of the disclosed bone substitute, consisting of hydroxyapatite, 2.5 wt % of a biocompatible glass and silicon and subsequently sintered at 1300° C. Each of the spectra refers to the material with different silicon contents, such as follows:

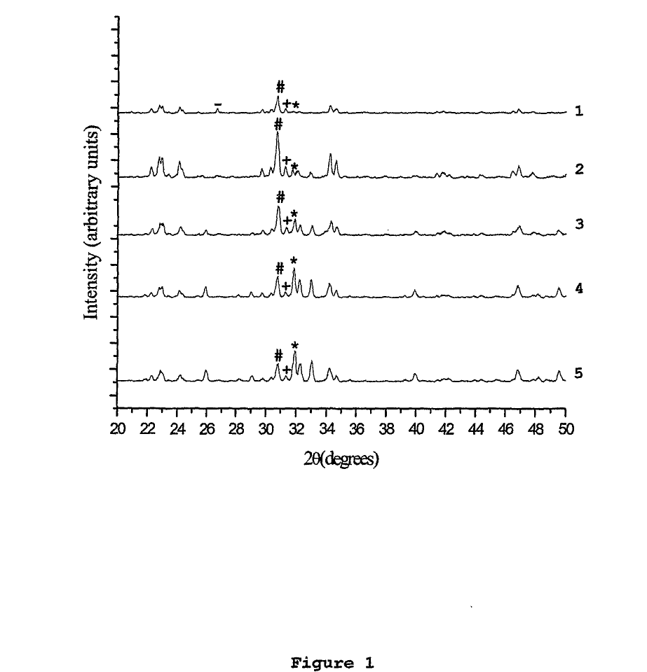

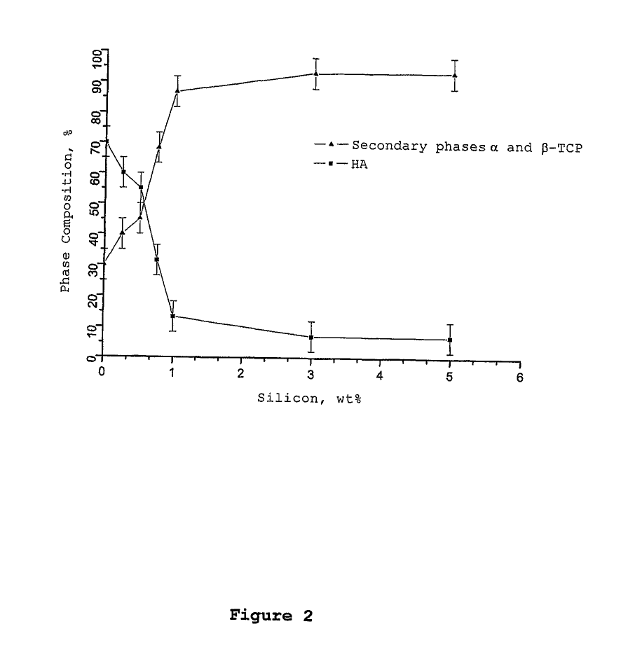

### 1—≧3.0 wt % Si

### 2—1.0 wt % Si

### 3—0.75 wt % Si

### 4—0.5 wt % Si

### 5—0.25 wt % Si

The symbols presented in the figure stand for:

### * Hydroxyapatite,

# alpha-TCP, + beta-TCP,

### − SiO₂ ou Ca₂SiO₄

FIG. 2—Phase quantification of the bone substitute consisting of hydroxyapatite, 2.5 wt % of a biocompatible glass and silicon, using colloidal silica as source, after sintering at 1300° C., wherein it is possible to observe the variation of alpha and beta-TCP with the amount of silicon added. Approximate phase quantification is determined through the ratio between the intensity of the three main peaks of hydroxyapatite and the three main peaks of the secondary phases, alpha and beta-TCP.

### DETAILED DESCRIPTION OF THE INVENTION

### 1. Hydroxyapatite Preparation

Hydroxyapatite is prepared by precipitation of the product resulting of the reaction between a calcium hydroxide (Ca(OH)₂, >98%) suspension in purified water and an aqueous solution of orthophosphoric acid 85 (wt/v) % H₃(PO₄)₂) according with the following chemical reaction:

Ca(OH)₂+6H₃(PO)₄→Ca₁₀(PO₄)₆(OH)₂+18H₂O

### 2. Glass Preparation

The biocompatible glass with nominal composition [60-75%]P₂O₅-[0-25%]CaO-[0-15%]Na₂O-[0-15%]CaF₂ (molar %) is prepared through a conventional melting process.

### 3. Bone Substitute Preparation

After the preparation of the abovementioned raw materials, they are milled and sieved under 75 μm. Afterwards, the biocompatible glass is added to hydroxyapatite in a weight percentage inferior to 10% relatively to hydroxyapatite weight.

This procedure is followed by the addition of silicon, in a percentage up to about 10 wt %, preferably up to 3 wt %, to hydroxyapatite and biocompatible glass, using conventional silicon sources, such as colloidal silica (silica nanoparticles), tetraethylorthosilicate (TEOS, Si(OC₂H₅)₄), tetrapropylorthosilicate (TPOS, Si(OC₃H₇)₄), silicon acetate (SiC₂H₃O₂) sodium silicate (Na₂SiO₃), calcium silicate (Ca₂SiO₄) or magnesium silicate (Mg₂SiO₄), among others. Then, dry or wet mixture is employed, for instance in a double cone mixer, in a planetary mixer or in a turbula, thus guaranteeing homogeneity.

During dry mixture process, the solid silicon source is directly added to the hydroxyapatite and biocompatible glass powders. Whereas wet mixture process uses aqueous or non-aqueous solvents depending on the silicon source employed and requires silicon solution or suspension preparation, with the appropriate concentration, which will be subsequently added to the hydroxyapatite and biocompatible glass powders. Under the circumstances of silicon solutions or suspensions preparation, the use of a conventional surfactant, such as methylcellulose, saponin, polyvinyl alcohol (PVA, [CH₂CHOH]ₙ), among others, is required.

Once the mixture is prepared, sinterization thermal treatment is performed, via gradual heating at a rate of 4° C./min until a temperature superior to 1100° C., preferably between 1200° C. and 1350° C., followed by a dwelling time at the chosen temperature, usually not inferior to 1 hour, and posterior natural cooling to room temperature inside the furnace.

### EXAMPLES

The example is provided for the purpose of a better comprehension of the disclosed invention, representing the preferred embodiments of the invention and is not intended to limit the scope of the invention.

### Example 1

### Synthetic Bone Preparation with Granular Format

### Hydroxyapatite Preparation:

100 g of hydroxyapatite are prepared by chemical precipitation according to the following chemical reaction:

10Ca(OH)₂+6H₃(PO)→Ca₁₀(PO₄)₆(OH)₂+18H₂O

In order to achieve that, 74.09 g of calcium hydroxide (Ca(OH)₂, >98%), 69.03 g of orthophosphoric acid 85 (wt/v) % (H₃PO₄) are weighed. Then, the calcium hydroxide is added to 1800 mL of purified water in a large container, and mixed (Mixer R25) during 15 minutes.

Meanwhile, orthophosphoric acid is added to 1600 mL of purified water in a beaker with 1800 mL capacity, and the volume is completed with purified water. The addition of orthophosphoric acid is performed via peristaltic pump (Minipuls 2) at a constant rate of 150 rpm.

The mixture is performed during 4-5 hours, and cleaning of the calcium hydroxide container walls with purified water is required in order to prevent precipitate accumulation. Throughout the process, a pH control using a 32% ammonia solution is performed in order to fix pH at 10.5±0.5. After the acid solution addition, the beaker is washed with purified water and the rate of the peristaltic pump is increased to 360 rpm.

Once the mixture is finalized, the solution in the container is mixed for 1 hour followed by a period of 16 hours where the mixture is left ageing.

Afterwards, hydroxyapatite filtration is performed and dried in a forced air circulation oven (Binder). Once dried, hydroxyapatite is milled in a planetary mill (Fritsch Pulverizette 6) and sieved under 75 μm.

### Glass Preparation:

Preparation of 0.2 mol of a glass with the following nominal composition 65% P₂O₅-15% CaO-10% CaF₂-10% Na₂O (molar %) are performed. In order to achieve that, 2.12 g of sodium carbonate (Na₂CO₃), 4.08 g of calcium hydrogenophosphate (CaHPO₄), 1.56 g of calcium fluoride (CaF₂) and 16.32 g of diphosphorus pentoxide (P₂O₅) are weighed and mixed in a platinum crucible.

The crucible is placed in a vertical oven (Termolab) which is heated during 1 h 30 min up to 1450° C., followed by a dwelling time of 30 minutes. After this period the molten glass is poured into purified water.

Once the glass is dry, it is milled in a planetary mill (Fritsch Pulverizette 6) and sieved under 75 μm.

### Bone Substitute Preparation:

A bone substitute with a silicon content of 1 wt %, is prepared by adding 106.2 mL of colloidal silica suspension 2 (wt/v) %, in purified water, to hydroxyapatite and biocompatible glass powders. In order to achieve a higher homogeneity of the wet mixture, 100 mL of purified water are added. Then, the mixture is placed in a turbula (TURBULA T2F) during a period of time not inferior to 1 hour, the mixture being subsequently dried in an oven (Binder). Once dried, the material is sieved under 75 μm.

Samples with a diameter of 30 mm are prepared by uniaxially pressing 5 g of the mixture powders at 288 MPa. Then, these samples are submitted to a sinterization thermal treatment performed via gradual heating at a rate of 4° C./min up to a temperature of 1300° C., followed by a dwelling time of 1 hour, and subsequent natural cooling to room temperature inside the furnace.

After sample sintering, milling in a planetary mill and sieving until final product granulometry are carried out inside a planetary mill (Fritsch Pulverizette 6) as well as a screening procedure until the final product's granulometry is obtained.

The silicon incorporation confirmation on the disclosed bone substitute is assessed by X-ray photoelectron spectroscopy (XPS). In the obtained Si 2p spectra, 101 eV binding energy suggests the occurrence of a PO₄³⁻ group substitution by SiO₄³⁻ group.

### REFERENCES

1. Carlisle E M, Silicon: A Requirement in Bone Formation Independent of Vitamine D1. Calcif Tissue Int, 1981. 33: p. 27-34.
2. Carlisle E M, Silicon: A Possible Factor In Bone Calcification. Science, 1970. 167(916): p. 279-80.
3. Milne D B Schwarz K, Growth-Promoting Effects of Silicon in Rats. Nature, 1972. 239: p. 333-334.
4. Vallet-Regi M, Revisiting Ceramics for Medical Applications. Dalton Trans, 2006. 44: p. 5211-5220.
5. Hastings G W Santos J D, Knowles J C, Sintered hydroxyapatite compositions and method for the preparation thereof, Patent Cooperation Treaty (PCT), World Intellectual Property Organization, 16 Nov. 2000.
6. Bonfield W Best M S, Gibson I R, Santos J D, Silicon-substituted apatites and process for the preparation thereof, Patent Cooperation Treaty (PCT), World Intellectual Property Organization, 5 Mar. 1998.
7. Smith T J N Pugh S M, Sayer M, Langstaff S D, Synthetic biomaterial compound of calcium phosphate phases particularly adapted for supporting bone cell activity, United States Patent, 25 Jan. 2005.

1-21. (canceled)

22. A composition comprising hydroxyapatite, biocompatible glass as a source of fluorine, and an independent silicon source-based bone substitute, comprising a mixture of hydroxyapatite, silicon in a concentration up to 10 wt % and biocompatible glass in a percentage up to 10 wt % relative to hydroxyapatite weight, said glass having the following nominal composition: 60-75% of P₂O₅; 0-25% of CaO; 0-15% of Na₂O; 0-15% of CaF₂ (% molar).

23. A composition according to claim 22 comprising silicon in a concentration up to 3 wt %, relative to hydroxyapatite and biocompatible glass weight.

24. A composition according to claim 22 further comprising a biocompatible polymeric vehicle selected from chitosan, dextran, hyaluronic acid, polylactic acid, poly(lactide-co-glycolic) acid or mixtures thereof.

25. A synthetic bone substitute or osteoregenerative product comprising a composition according to claim 22 wherein the bone substitute or osteoregenerative product, without the presence of a glassy phase on its structure, has a higher proportion of alpha and beta-TCP secondary phases caused by the addition of amounts of silicon.

26. A synthetic bone substitute or osteoregenerative product according to claim 25 wherein the synthetic bone substitute is presented as granules, three-dimensional pieces, custom-made implants, prostheses, implant coatings, or bone cements.

27. A composition according to claim 22, wherein said composition is associated with a drug controlled release system or with stem cells.

28. A process for the preparation of a composition comprising hydroxyapatite, biocompatible glass as a source of fluorine and an independent silicon source-based bone substitute, wherein a mixture of the silicon source with hydroxyapatite and biocompatible glass is sintered, at a temperature higher than 1100° C. and lower than 1350° C., thereby causing glass melting and fusion throughout the hydroxyapatite structure, resulting in a triphasic product of hydroxyapatite [Ca₁₀(PO₄)₆(OH)₂], alpha and beta tricalcium phosphate [α- and β-Ca₃(PO₄)₂] and dispersed silicon in the structure without the presence of a glassy phase after sintering.

29. A process according to claim 28 wherein the sintering is carried out at a temperature range of 1200-1350° C.

30. A process according to claim 28 wherein a silicon content increase implies a reduction of the hydroxyapatite percentage and consequent increase of secondary phases alpha and beta-TCP percentage.

31. A process according to claim 28 wherein the independent silicon source furnishes a silicon addition up to about 10 wt % in which the silicon added in equal or superior amounts to 3 wt % results in an additional silicon-containing secondary phase appearance besides alpha and beta-TCP.

32. A process according to claim 31 wherein the additional silicon-containing secondary phase is silica (SiO₂) and/or a calcium silicate.

33. A process according to claim 28 wherein the silicon source is mixed with the hydroxyapatite and biocompatible glass, before sintering, during any step of the preparation process, and wherein the silicon source is selected from the group of the colloidal silica type, tetraethylorthosilicate (TEOS, Si(OC₂H₅)₄), tetrapropylorthosilicate (TPOS, Si(OC₃H₇)₄), silicon acetate (SiC₂H₃O₂), sodium silicate (Na₂SiO₃), calcium silicate (Ca₂SiO₄), magnesium silicate (Mg₂SiO₄), and mixtures thereof.

34. A process according to claim 28 wherein mixture of the silicon source with hydroxyapatite and biocompatible glass is performed by a dry or wet process wherein, in the case of a dry mixture, the solid silicon source is directly added to the hydroxyapatite and biocompatible glass powders, and wherein, in the case of a wet mixture, aqueous or non-aqueous solvents are employed during the preparation of a silicon solution or suspension which is subsequently added to the hydroxyapatite and biocompatible glass powders.

35. A process according to claim 34 wherein the dry mixture process requires direct addition of the solid silicon source with the hydroxyapatite and biocompatible glass powders, and wherein in the wet mixture process silicon solutions or suspensions preparation requires the use of a surfactant to produce homogeneous silicon distribution on the bone substitute.

36. A process according to claim 34 wherein the mixture of hydroxyapatite, a biocompatible glass and silicon is obtained by means of liquid phase sintering above 1100° C., resulting in the formation of a distinguishable microstructure of three crystallographic phases, hydroxyapatite and the secondary phases alpha and beta-TCP, in different proportions according to the amount of silicon added, resulting in a unique microstructure with enhanced mechanical properties.

37. A process according to claim 28 wherein the temperature range results in glass melting and diffusion within the hydroxyapatite structure leading to the occurrence of several network ionic substitutions and dispersed silicon.

38. A process according to claim 28 wherein, once the mixture is prepared, sintering is performed, via gradual heating at a rate of 4° C./min until a temperature superior to 1100° C. is achieved, followed by a dwelling time at the chosen temperature for at least 1 hour, and posterior natural cooling to room temperature inside the furnace.

39. A process according to claim 28 wherein mixing is performed, before sintering, during any step of the preparation process, through dry or wet route.
